(19) United States
(12) Patent Application Publication
(10) Pub. No.: US 2015/0106111 A1
(43) Pub. Date: Apr. 16, 2015

(54) SYSTEM, METHOD AND COMPUTER PROGRAM PRODUCT FOR DIAGNOSTIC AND TREATMENT ENHANCEMENT

(71) Applicant: Sage Health Management Solutions, Inc., Minneapolis, MN (US)

(72) Inventor: V. Katherine Gray, Minneapolis, MN (US)

(21) Appl. No.: 14/336,473

(22) Filed: Jul. 21, 2014

Related U.S. Application Data
(60) Provisional application No. 61/889,300, filed on Oct. 10, 2013.

(51) Int. Cl. G06F 19/00; G06N 5/00; G06F 17/30

(52) U.S. Cl. CPC G06F 19/345; G06F 17/30867; G06N 5/003
USPC 705/2

(57) ABSTRACT

Diagnostic enhancement procedures involve identifying a complete or substantially complete diagnosis code for a patient in response to receiving a query related to a search for a patient diagnosis code followed by entry of patient clinical criteria by a user, such as a clinician. The query may include entry of a procedure and/or procedure code the results of which the user suspects will result in a complete diagnosis and/or diagnosis code. The complete or substantially complete diagnosis code is used in combination with the patient's clinical criteria selections to generate a procedure recommendation and corresponding procedure code. A parent or child diagnosis code is identified and used to select an appropriate procedure recommendation decision tree, where branches of the decision tree are assigned a particular parent or child code, and patient clinical criteria selections are used to narrow the diagnostic recommendation to a specific procedure and corresponding code.

### CROSS-REFERENCE TO RELATED APPLICATION

The present application claims priority to U.S. Provisional Application Ser. No. 61/889,300, filed Oct. 10, 2013, the entire contents of which are hereby incorporated by reference in their entirety.

### TECHNICAL FIELD

Systems, methods and computer program products are provided for determining diagnosis and recommending procedure codes, and in more specific implementations, may be used to determine ICD-10 CM and recommend CPT and/or ICD-10 PCS codes.

### BACKGROUND

As required by the Patient Protection and Affordable Care Act of 2010, the use of standard diagnostic and procedure codes changes to a new standard that, as of the filing date of the present application, is expected to go into effect on Oct. 1, 2015. This new standard is used globally except in the U.S. and will greatly improve comparable measures to other countries for health care indices and outcomes. The new code set from the World Health Organization (WHO), which is referred to as International Statistical Classification of Diseases and Related Health Problems (version 10), has been modified in the U.S. with over 68,000 diagnosis (ICD-10 CM) codes and 76,000 procedure (ICD-10 PCS) codes for use in hospital inpatient settings. The new coding system is radically different than the previous diagnosis array and much more detailed.

With the introduction of ICD-10 CM and PCS codes, U.S. practitioners must be able to quickly and easily find the proper category of diagnosis for the patient, document details that will be needed for additional required refinement of the diagnosis code that must be used for billing by the performing practitioner, and document the key leaf node decisions that reflect the evidence that has been collected about what issues define the most beneficial diagnostic testing to determine the proper diagnosis for a patient. With the proper diagnosis, then treatment can begin for the patient.

### SUMMARY

The present disclosure addresses these new requirements to assist the practitioner in determining the proper diagnosis code and the proper diagnostic procedure. Particularly, in view of the larger, more complicated diagnosis data set, more detail about the patient is needed in order to assign the proper diagnosis code and hence provide a recommendation for the proper diagnostic testing, and a decision making tool to enhance the diagnosis and procedure recommendation process is provided herein. In addition, once the proper diagnosis is identified, an enhanced treatment recommendation routine may be provided.

According to one implementation, a computer-implemented method for recommending a procedure involves using a computer processor to perform the steps of: receiving at least a partial diagnosis code; using the at least partial diagnosis code to identify a plurality of clinical criteria associated with the at least partial diagnosis code, the plurality of clinical criteria including a plurality of signs and symptoms and stored in a computer memory; receiving one or more selections of the plurality of clinical criteria; generating a diagnosis code or a substantially complete diagnosis code using the at least partial diagnosis code and the received one or more selections of the plurality of clinical criteria, where the diagnosis code or substantially complete diagnosis code includes a greater number of digits relative to the at least partial diagnosis code; identifying one diagnostic procedure recommendation decision tree from a plurality of diagnostic procedure recommendation decision trees stored in the computer memory using the generated diagnosis code or substantially complete diagnosis code, where branches of the decision tree are each assigned a different partial diagnosis code, and decision end points of the decision tree are each associated with a different procedure recommendation and corresponding procedure code; determining a set of clinical criteria required to recommend a procedure using the decision tree and the selections of the plurality of clinical criteria; providing additional clinical criteria for selection using the determined set of clinical criteria; receiving selections from the provided additional clinical criteria selections, the selections associated with a patient's signs and symptoms; and providing a recommended procedure to administer to the patient and a corresponding procedure code by applying the received selections of additional clinical criteria to the identified decision tree.

In another implementation, a computer-implemented method for diagnostic enhancement involves receiving a query related to a search for a patient diagnosis code, identifying a plurality of partial diagnosis codes based on the received search, receiving a selection of a partial diagnosis code. The method continues by using the partial diagnosis code to identify a plurality of signs and symptoms associated with the partial diagnosis code. Based on selections of one or more of the signs and symptoms, a complete diagnosis code or a substantially complete diagnosis code (i.e., a diagnosis code with a greater number of digits compared to the partial diagnosis code) is generated. The diagnosis code is used in combination with the received patient signs and symptoms to determine a recommended procedure and corresponding procedure code. Where the diagnosis code is substantially complete, generating the recommended procedure may involve identifying the proper procedures to confirm diagnosis and identify a corresponding complete diagnosis code.

In a more particular implementation, a computer-implemented method for recommending a procedure involves using a computer processor to perform the steps of: receiving a query for a search for a diagnosis code; identifying a plurality of at least partial diagnosis codes, stored in a computer memory, using the received query; receiving a selection of one of the at least partial diagnosis code from the plurality of at least partial diagnosis codes; identifying a plurality of clinical criteria related to the selected at least partial diagnosis code, the plurality of clinical criteria comprising a plurality of signs and symptoms; receiving one or more selections of the identified plurality of clinical criteria; generating a diagnosis code or a substantially complete diagnosis code using the selected at least partial diagnosis code and the received one or more selections of the plurality of clinical criteria, wherein the diagnosis code or substantially complete diagnosis code comprises a greater number of digits relative to the selected at least partial diagnosis code; and providing a recommended procedure and corresponding procedure code using the generated diagnosis code or substantially complete diagnosis code and the received selections of the plurality of clinical criteria.

In yet another implementation, a complete or partial diagnosis code may be provided, for example, from patient records, and used to identify an appropriate procedure recommendation decision tree, where branches of the decision tree are assigned a partial diagnosis code, and decision end points are associated with procedure recommendations arrived at by matching patient clinical criteria entered by the user. Particularly, a plurality of signs and symptoms for the patient clinical criteria associated with the diagnosis code are provided to a user for selection; and received selections are used to identify procedure recommendations.

In a more particular implementation, a computer-implemented method for recommending a procedure involves using a computer processor to perform the steps of: receiving at least a portion of a diagnosis code; identifying one diagnostic procedure recommendation decision tree from a plurality of diagnostic procedure recommendation decision trees stored in a computer memory using the received at least a portion of a diagnosis code, where branches of the decision tree are each assigned a different partial diagnosis code, and decision end points of the decision tree are each associated with a different procedure recommendation and corresponding procedure code; determining a set of clinical criteria required to recommend a procedure using the identified decision tree; providing clinical criteria selections using the determined set of clinical criteria; receiving selections of clinical criteria associated with a patient from the provided selections; and providing a recommended procedure to administer to the patient and a corresponding procedure code by applying the received selections of clinical criteria to the identified decision tree.

In yet another implementation, once a diagnosis code is complete, for example, based on executing the processes of the above implementations, the diagnosis code and the patient's clinical criteria entered by the user may be used to enhance treatment recommendations for the patient.

### BRIEF DESCRIPTION OF THE DRAWINGS

FIG. 1 illustrates a diagnostic enhancement system according to the present disclosure.

FIG. 2 illustrates a flow diagram of a method of providing a recommended procedure according to the present disclosure.

FIG. 3 illustrates a flow diagram of a method of identifying a complete or substantially complete diagnosis code.

FIG. 4 illustrates a flow diagram of a method of providing a procedure recommendation.

### DETAILED DESCRIPTION

Overview

In some aspects, diagnosis and procedure codes are obtained based on identifying a partial diagnosis code in response to search criteria input by a user, which is followed by an iterative process of receiving information about the patient's signs and symptoms, i.e., patient clinical criteria. This information is used to identify a more complete diagnosis code and to generate a procedural task recommendation. The resulting diagnosis code may include the largest number of digits possible for what is known about the patient prior to diagnostic testing. This diagnosis code and patient-specific information may also be used in identifying procedure codes (either CPT or ICD-10 PCS) that are the most appropriate for the patient. Although the present disclosure may be particularly suitable for identifying proper ICD-10 CM and PCS codes for determining the proper procedure to confirm a diagnosis, implementations may be used to identify other types of recommendations that rely on the medical diagnosis and procedure codes.

In a first scenario, a clinical practitioner may enter a general clinical term in the form of a “phrase” or “keyword” based upon their expertise to select the most appropriate diagnosis. In this first scenario, entry of the general clinical term results in display of high level diagnosis code choices, such as 3- or 4-digits of ICD-10 CM codes, and after a high level code selection is received, the practitioner may then review displayed symptoms and signs to allow the practitioner to enter selections that relate to the patient's symptoms and/or signs. These selections may be used both to impute a more specific code and to provide the most specific criteria for determining a procedure recommendation and corresponding procedure code, e.g., CPT or ICD-10 PCS code.

Accordingly, aspects of the present disclosure separate the need for information about which diagnosis (ICD-10 CM) code should be documented from the need for information to recommend a diagnostic procedure code (either CPT or ICD-10 PCS code).

In a second scenario, the practitioner may have a specific code, for example, from another electronic source (e.g. electronic health record (“EHR”), electronic medical record (“EMR”), or Patient Questionnaire (“PQ”)). In cases where the starting code is more specific, such as 5-, 6-, or 7-digits of ICD-10 CM code, then the symptoms and signs pertaining to the particular leaf node may be displayed for documentation and to enable the user to confirm that the patient should be offered the recommended procedure.

In additional aspects of the present disclosure, practitioners may be assisted in documenting the coding process with greater granularity by using their understanding of the clinical characteristics of patients but without having to know the details of coding. This may provide more efficient and accurate coding processes and may reduce administrative costs. Furthermore, the greater specificity provided by the coding processes may assist in selecting the most appropriate procedure without additional work by clinical or administrative staff. Accordingly, the present disclosure adds modernization to diagnostic enhancement technology.

Implementations may address instances where there is no “known” diagnosis, and methods may involve receiving patient information that is preliminary and piecemeal. With preliminary information, a recommendation is needed to narrow the inappropriate ordering of diagnostic tests, while giving the patient and healthcare practitioner the most likely scenario for getting to an accurate diagnosis. In prior approaches, evidence-based medical literature has been used to guide the decision making by the practitioner with both transparency and flexibility, and is disclosed in U.S. Pat. No. 6,149,585, entitled Diagnostic Enhancement Method and Apparatus, issued on Nov. 21, 2000, including a common inventor, and which is incorporated by reference in its entirety for any useful purpose.

System Overview

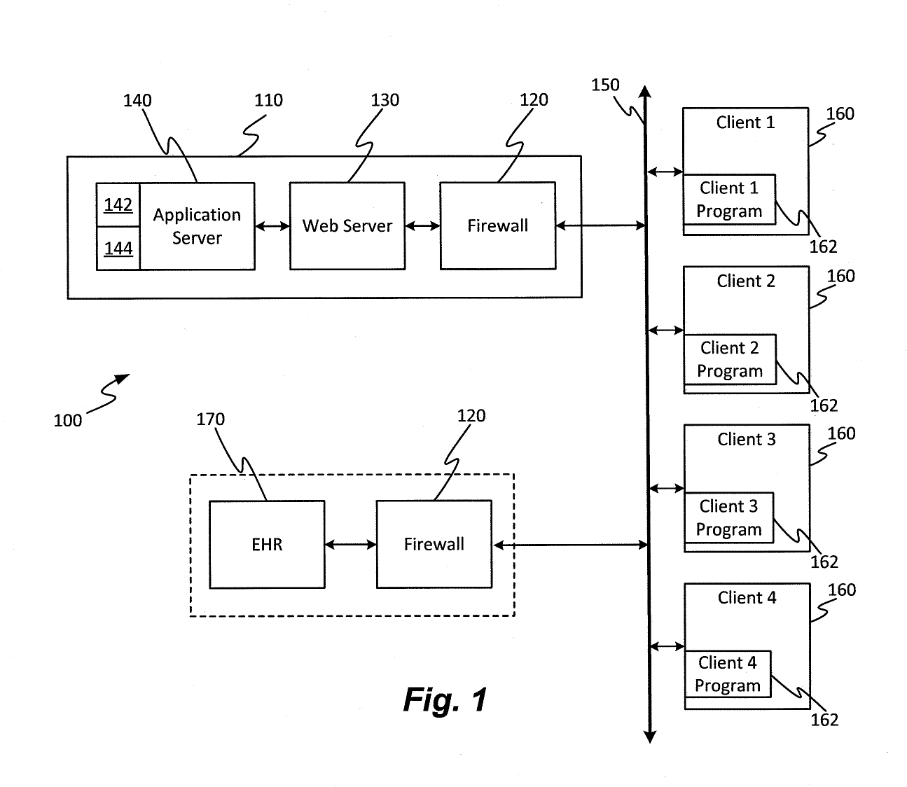

FIG. 1 illustrates an exemplary diagnostic enhancement system 100 according to the present disclosure. According to FIG. 1, the diagnostic enhancement system 100 may be implemented using the Internet, but may additionally or alternatively be implemented using other various communication systems including local area networks (“LANs”), wide area networks (“WANs”), may be integrated with EHR/EMR systems for example via web services or a software development kit (“SDK”), and so on. System 100 includes a server system 110 communicatively coupled with a plurality of clients 160 and an electronic health record system 170 containing electronic health record (“EHR”) data via a network communication interface 150. Although EHR data is described in connection with the diagnostic enhancement system 100, other types of patient data such as EMRs, PQ, or other clinical or self-reported patient information may be used as input for services executed by the system 100, such as diagnostic enhancement routines.

The server system 110 may include a firewall 120 interposed between the network communications interface 150 and a web server 130 linked to an application server 140. The web server 130 may be in communication with the application server 140. The application server 140 includes instructions for a diagnostic enhancement program 142 and database/database program 144. Each of the clients 160 includes a client program 162 (e.g., any suitable Internet browser) to enable a user to communicate with the diagnostic enhancement program 142.

Firewall 120 may be any suitable device (or software in a router) that securely links the server system 110 (e.g., an organization's internal TCP/IP network) to the Internet or other communication system associated with the clients 160. In addition, firewall 120 may require users to log in to the server system 110 for obtaining access to the diagnostic enhancement program 142.

The web server 130 of the server system 110 may be a conventional web server and may allow the diagnostic enhancement program 142 to be accessed over the Internet. The web server 130 may receive incoming requests from clients 160 and transmit appropriate documents (e.g., HTML, J-Script documents). The web server 130 may be implemented with any suitable computer executing an appropriate web server program. The web server 130 may be replaced with, or be used in combination with, other network servers adapted to provide services over a LAN or WAN.

The diagnostic enhancement application server 140 of the server system 110 may be implemented with any suitable computer for executing the diagnostic enhancement program 142 and the database/database program 144. The purpose of the diagnostic enhancement program 142, in connection with the database/database program 144, is to perform a diagnostic enhancement routine, described herein, in order to assist and monitor users in determining proper diagnosis and procedure codes. The diagnostic enhancement program 142 may be implemented with any suitable server side (or even client side, e.g., JavaScript) application scheme using an adequate programming structure and language. For example, the diagnostic enhancement program 142 could be implemented with a common gateway interface (“CGI”) script application. Likewise, any suitable database program may be used to implement the database/database program 144.

Client workstation 160 may be any suitable computer for sending and receiving data over the network communications interface 150. Client program 162 of the client workstation 160 may facilitate the integration of services from the server system 110 with services from existing electronic health record system 170. In one scenario, the client program may be a standard Internet browser used to communicate with the server system 110 and the electronic health record system 170. For example, components of the diagnostic enhancement system 100 may communicate with the electronic health record system 170 by HL7 or other data exchange formats to make available patient demographics to be used in connection with executing functions of the diagnostic enhancement program 142 and the database/database program 144. Alternately, the diagnostic enhancement system 100 may call any available application programming interface (“API”) to access the electronic health record system 170 directly to obtain patient demographic information and send diagnosis and treatment information back thereto for updating the patient EHRs. In another scenario, the client program 162 may include a dynamic link library (“DLL”) adapted to communicate with the electronic health record system 170 through the client workstation 160. Using one or more of these various communication protocols, the client program 162 may operate to retrieve patient information from EHRs during an encounter and record procedures to be performed while avoiding redundant entries by the user of the client workstation 160.

Although the diagnostic enhancement system 100 of FIG. 1 depicts only one server system 110, more than one server system 110 may be used, depending upon particular network requirements. For example, multiple redundant servers could be implemented for both faster operation and enhanced reliability. Also, additional servers may be used for various alternative functions (e.g., as a gateway) within server system 110.

Diagnostic Enhancement Routine

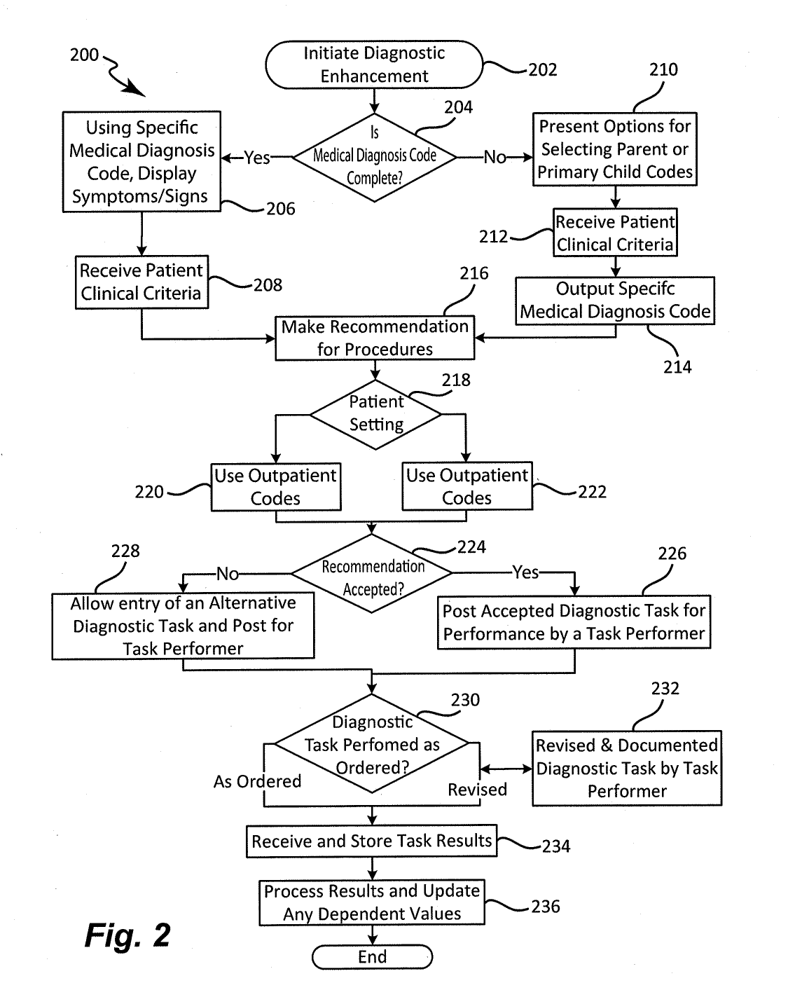

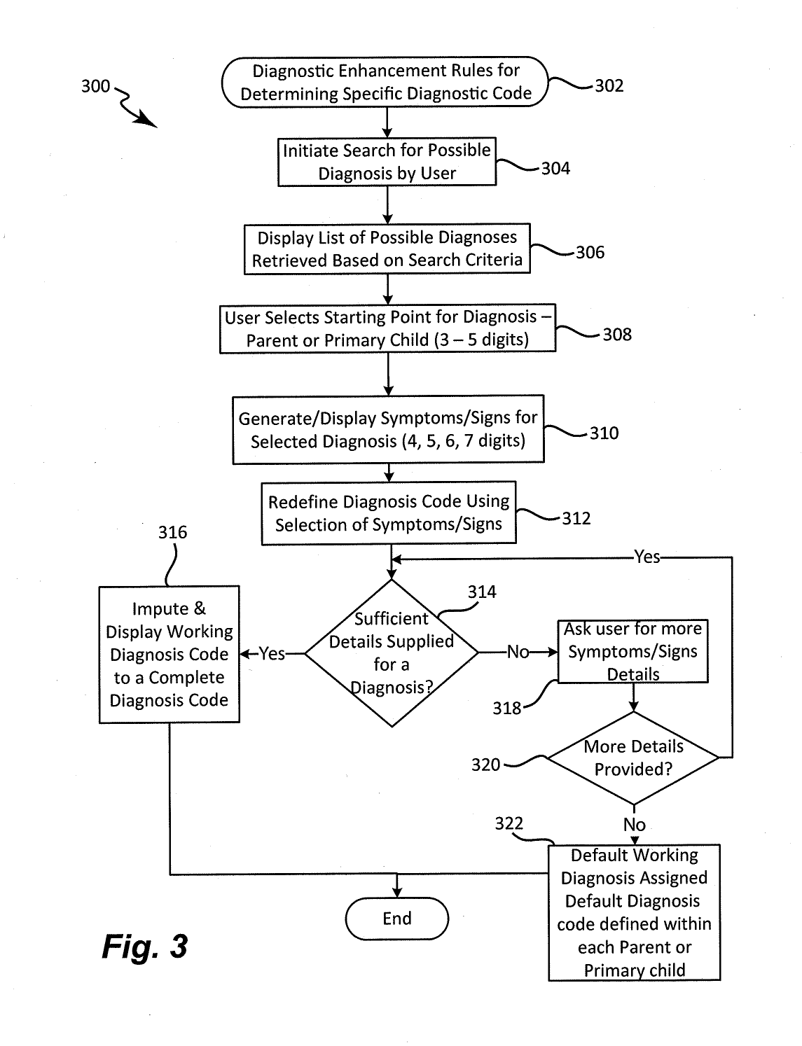

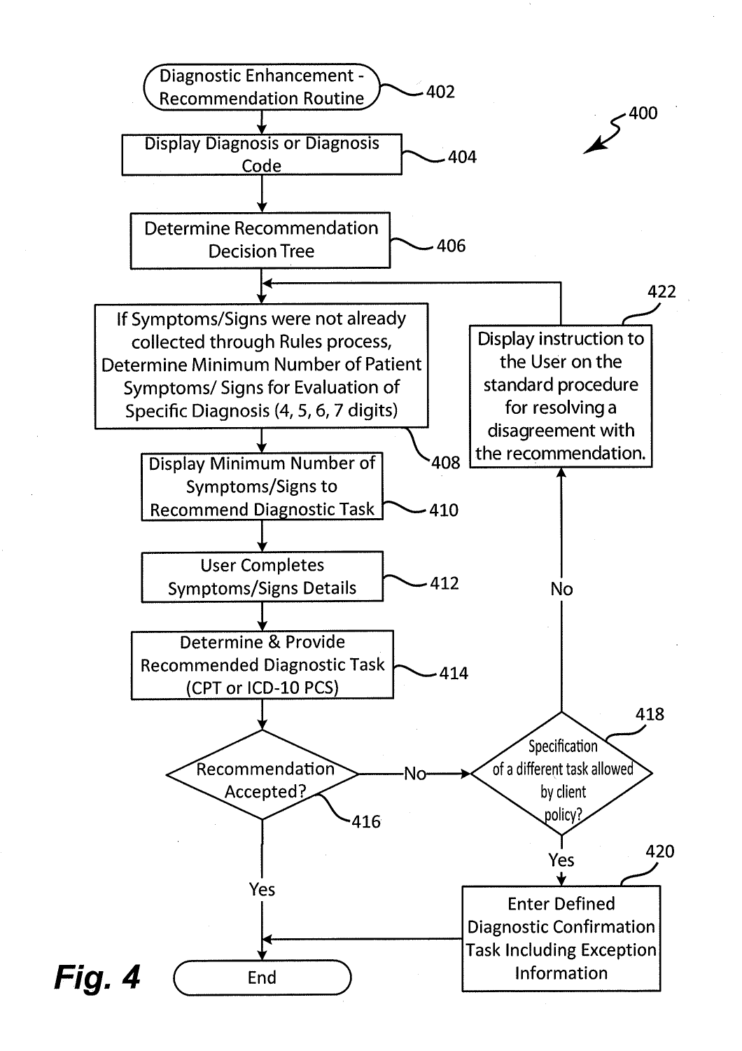

Turning to FIG. 2, a flowchart illustrating a method 200 for implementing a diagnostic enhancement routine is provided. The method may be initiated at operation 202 following the generation of a diagnosis code according to the process described in connection with FIG. 3, or the generation of a recommended diagnostic task (e.g., procedure code) according to the process described in connection with FIG. 4, following both the FIG. 3 and FIG. 4 processes, or portions thereof. Particularly, the user (e.g., a physician, nurse practitioner, allied practitioner, physician assistant, etc.) may use the diagnostic enhancement technology of the present disclosure to determine the proper diagnosis code (e.g., according to the process of FIG. 3) and most appropriate procedure code (e.g., according to the process of FIG. 4) for either inpatient or outpatient settings.

Continuing with FIG. 2, a determination of whether the medical coding, e.g., the ICD-10 coding process, is complete (e.g., by diagnosis from another electronic system) is made in operation 204. If so, the specific code is utilized to retrieve symptoms and signs associated with the code in operation 206, and the signs and symptoms may be displayed for selection by the user. More particularly, if the user has a complete ICD-10 CM code (usually 6- or 7-digits), the system will display the specific patient clinical criteria selections for that refined ICD-10 CM code in order for the user to document the criteria that are present for the patient. This documentation assures that the patient “qualifies” for the refined code in terms of meeting the evidence-based guidelines for that procedure recommendation. Operation 206 may additionally involve using a user-entered procedure or procedure code to generate and display signs and symptoms for selection by the user. In this example, the user may enter selections that pairs the diagnosis code with the procedure/procedure code the user suspects should be performed. Patient clinical criteria displayed may be associated with the diagnosis or aspects of the diagnosis which the user-entered procedure is intended to identify. In operation 208, the aforementioned patient clinical criteria selections are received.

Where it is determined that the medical coding process is not yet complete in operation 204, then the flow proceeds to operation 210 where options for selecting parent or primary child diagnosis codes are displayed. Operation 210 may additionally involve displaying one or more fields for a user to enter a procedure or procedure code the user suspects should be performed to complete a diagnosis. In some cases, the partial diagnosis code and procedure/procedure code may be paired and the clinical criteria may be displayed based on the pairing. In operation 212, patient clinical criteria selections are received upon entry by the user. More particularly, in this operation, the system will display patient criteria to be marked if present. Once all the patient information is input, the process flows to operation 214 where the most specific diagnosis code, e.g., ICD-10 CM code is output (e.g., to the most granular level possible).

From either operation 208 or operation 214, the process flow continues by generating a recommendation for a procedural task in operation 216. Where the user enters a procedure (e.g., in operation 210), the procedural task recommended may differ from the entered procedure due to the recommendation being generated based on the clinical criteria selections. In operation 218, the process may receive a patient setting selection of either outpatient or inpatient. Depending on the selection, recommended outpatient codes (e.g., CPT codes) are presented for outpatient situations in operation 220, or recommended inpatient codes (e.g., ICD-10 PCS codes) are presented for inpatient settings in operation 222. Alternatively, the user may see the outpatient code(s) (e.g., CPT codes), inpatient code(s) (e.g., ICD-10 PCS codes) or both as the recommendation. Once a recommendation is made, the user selects whether to accept the recommendation.

In operation 224, a determination is made about whether the procedural task recommendation is accepted. Where accepted, the process continues to post (e.g., record) the accepted diagnostic task for performance by a performing practitioner (e.g., diagnostic testing professional such as a radiologist or a lab technician) in operation 226. Where the recommended task is not accepted, the process continues by allowing the performing practitioner to enter an alternative diagnostic task and posting of the entered tasks for performance by the performing practitioner in operation 228. Particularly, the performing practitioner may document an alternate procedure and provide information about why the patient requires an exception. These exception data may provide information for improvement to the recommendations engine and the clinical content database.

In operation 230, a determination is made about whether the diagnostic task was performed as ordered. If not, the process flow may involve entry of task revisions and documentation by the performing practitioner (either real time or by later messaging) in operation 232. If the diagnostic task was performed as ordered and results created (i.e. interpretation or report), or after operation 232, the process flows to operation 234 where a storage device receives and stores task results (e.g., either real time or by later messaging). In operation 236, the results are processed and dependent values are updated, such as the categorization of the reports into findings of either positive, negative, or equivocal (or more complicated findings sub-categories). The process of FIG. 2 may end following operation 236.

Diagnostic Enhancement Routine: Diagnosis Code Determination

Turning to the flowchart of FIG. 3, a method 300 for determining a diagnosis code (e.g., ICD-10 CM code) is illustrated. As described above, the method of FIG. 2 may be modified according to the method depicted in the flowchart of FIG. 3, or portions thereof. According to FIG. 3, a diagnostic enhancement routine for determining a specific diagnosis code is initiated at operation 302. In operation 304, a user engages in a search for a possible diagnosis by entering search criteria (e.g., keywords, Boolean phrases, medical codes, body systems). In some implementations, search criteria entered may include a procedure or procedure code where the user suspects the outcome of such procedure will result in determining a diagnosis. Particularly, if the user does not have a specific code for a diagnosis, the user may initiate a search using search terms for identifying a diagnosis code starting point. This starting point may be referred to as a parent code (typically 3-digits in ICD-10 CM) or may be a primary child code (typically 4- or 5-digits in ICD-10 CM). A parent code has child codes that use additional digits (4, 5, 6, or 7) to fully describe the specific diagnosis and, in general, a parent code will allow the user to define as much as they can to output the most specific diagnosis code. On the other hand, a primary child code is complete and can be used as it is.

In operation 306, a list of possible diagnoses is retrieved and displayed based on the entered search criteria. The possible diagnoses may be displayed as starting codes bearing an association with the entered search criteria. The starting diagnosis codes may be parent or primary child codes, for example, having between 3- and 5-digits. In operation 308, the user selections of the starting codes may be received as a starting point for generating a complete or substantially complete diagnosis code. In response to receipt of the selection corresponding to the starting code, symptoms and signs for the selected diagnosis code may be displayed in operation 310. For example, once the starting diagnosis is selected, a display is generated of the symptoms and signs, including previous exams, contraindications, safety issues, and body parts that are needed for both proper documentation and choice for the most appropriate procedure to be recommended. In some implementations, the starting code selected and a procedure/procedure code entered by the user may be paired to result in generation and display of the clinical criteria for selection by the user. In operation 312, user selections of patient clinical criteria result in further refinement of the diagnosis code, e.g., up to 4, 5-, 6- or 7-digits. As the number of digits in the diagnosis code increases, the complexity of the diagnosis increases, and the user may be presented with signs and symptoms with increasing specificity with increased diagnosis complexity. In addition to refining the diagnosis code from the selections, these may be used for documentation of the patient's condition and for generating a procedure recommendation, described below.

In operation 314, a determination is made as to whether sufficient details have been submitted for determining a diagnosis. This determination may be based on whether the diagnosis code can be completely or substantially identified based on the received selections. In some instances, the diagnosis code may be sufficiently complete even when the code includes incomplete digits, e. g., where a digit is missing or the code includes a wildcard or underscore placeholder, particularly when a procedure is required to be performed in order to complete the digit within the diagnosis code. In cases where the code is incomplete, a default value may be assigned to this portion of the code. In some cases, pre-defined rules may be applied to determine when digits of an incomplete diagnosis code can be assigned default values and when more specific information is needed.

If sufficient details have been supplied to determine a diagnosis in operation 314, the flow proceeds to operation 316 where a working diagnosis code (e.g., ICD-10 CM) is imputed to a complete diagnosis code and displayed. The working diagnosis code is the most complete diagnosis code available. Particularly, using the patient criteria entered by the user, the most complete diagnosis code, e.g., with 5-, 6-, or 7-digits in the ICD-10 CM, is imputed. As mentioned, in certain cases, some digits of the diagnosis code may be assigned default values, which may be updated in response to results of a diagnostic procedure, the procedure having been recommended according to the present disclosure. With this information about the diagnosis code, the system can then move to the recommendation of a procedure.

If the details of the patient's diagnosis are not sufficient to determine a diagnosis code in operation 314, then the user is presented with relatively more detailed symptoms and signs for selection in operation 318. In operation 320, a determination is made as to whether more details have been provided in response to the presentation. If not, a default working diagnosis is assigned in operation 322. In some cases, the default diagnosis code may be one that is associated with a parent or primary child code selected in operation 308.

Returning to operation 320, if additional details have been provided in operation 318, the flow returns to operation 314 where it is determined whether the additional details are sufficient for a diagnosis to be generated. If not, the flow proceeds back to operation 318, with the option of the flow repeating the operations 318 and 320.

Following operation 316 or operation 322, the process of FIG. 3 may end.

As default values may be assigned to a diagnosis code, implementations accommodate incomplete diagnosis codes such as incomplete ICD-10 CM codes. For example, some digits of the code may depend on findings of diagnostic tests, and digits related to unknown findings may be left as default values. However, even with default values assigned to a diagnosis code, implementations provide a diagnostic basis, derived from user input related to the patient's clinical criteria, for use in identifying appropriate procedure recommendations and procedure code recommendations. Accordingly, and as described below, appropriate procedure recommendation decision trees may be implemented even in the absence of a complete ICD-10 CM code, namely, based on the combination of the partial code and selections entered by the user. In addition, the digits designated as the most general, default case, may be output if more specific information is not provided. For example, if a user refuses to input detailed information in response to the displayed symptoms and signs, they may select a default data set for the starting point to use for processing and ordering diagnostic procedures.

Diagnostic Enhancement Routine: Procedure Code Recommendation

Turning to the flowchart of FIG. 4, a method 400 for recommending a diagnostic procedure and corresponding procedure code (e.g., CPT or ICD-10 PCS code) is illustrated. As described above, the method of FIG. 2 may be modified according to the method 400 depicted in the flowchart of FIG. 4, or portions thereof. In operation 402, a diagnostic enhancement-recommendation routine may be initiated. The flow proceeds to operation 404 where a diagnosis and/or diagnosis code may be displayed. This information may be imputed from the method of FIG. 3, from a diagnosis code retrieved from a patient's electronic records, e.g., EHR, EMR, PQ, may be entered by a clinician, or combinations. Accordingly, the diagnosis code may be a partial diagnosis code with placeholders as described above. In addition, operation 404 may involve displaying a procedure code or procedure, which may be imputed from the patient's electronic records, may be entered by a clinician, or combinations. The procedure and/or procedure code may be one the user suspects will result in a complete diagnosis code. Where the procedure code is displayed, the user may enter a selection which pairs the procedure with a diagnosis and/or corresponding suspected diagnosis code or the system may automatically pair the diagnosis and procedure codes based on user selections. In operation 406, the flow proceeds by identifying a procedure recommendation decision tree to implement based on the available diagnosis or diagnosis and procedure information. For instance, the decision tree may be selected based on the parent or primary child diagnosis code first 3-5 digits or may be selected based on the entered procedure. Branches of the decision tree are each assigned a different partial diagnosis code, and decision tree end points are each associated with a different procedure recommendation. Using any available user-entered information of the patient's clinical criteria, e.g., signs and systems, various decision points in the decision tree are executed thereby arriving at a decision tree end point. Where the diagnosis code is a partial diagnosis code, one or more procedures related to identification of the correct digit(s) in the code, e.g., to render a more complete diagnosis code, may be evaluated in connection with the user-entered information. If the information gathered in operation 404 is derived from the method from FIG. 3, or portions thereof, the detailed patient symptoms and signs may already be of record and applied to the decision tree. In operation 408, the least number of symptoms and signs possible may be determined in order to evaluate the specific diagnosis code. This is done by identifying only the symptoms and signs associated with that specific recommendation branch of the decision tree. In operation 410, the flow continues by displaying the minimum number of symptoms and signs to recommend a diagnostic task for the proper procedure recommendation. In operation 412, the user completes the questions presented by entering patient clinical criteria. Based on the user input, operation 414 proceeds by providing a recommended diagnostic task and code, e.g., CPT or ICD-10 PCS code. The recommendation is based on applying the clinical criteria selections to the decision tree to narrow the diagnostic down to a decision tree end point having an associated procedure recommendation and corresponding procedure code. Accordingly, generating a procedural task recommendation involves use of the patient's clinical criteria related to signs and symptoms of the diagnosis, which is in addition to the patient's diagnosis code. Further, by evaluating the information required to complete a diagnosis code in connection with this process of identifying a procedure code, implementations may be particularly useful for confirming a diagnosis and completing a corresponding diagnosis code. Using this additional patient documentation improves both appropriate utilization and standardizes care, and provides data for trend analysis and process improvement. In some implementations, where the received selections of clinical criteria in operation 412 are insufficient to reach a decision tree end point, operation 414 may proceed by providing a default diagnostic task recommendation using the diagnosis code or partial diagnosis code identified in operation 404.

In operation 416, a determination is made about whether the recommended diagnostic task was accepted. If so, the flow may end. If not, the flow proceeds to operation 418 where a determination is made about whether a different procedural task is specified and allowed by the patient's insurance policy or health care provider ordering process. If so, the flow proceeds to operation 420 where a user may enter a defined diagnostic confirmation task and may include exception information, and the flow may end. If not, the flow proceeds to operation 422 where the user is instructed about standard procedures for resolving a disagreement with a recommendation, and the user may be prompted to re-enter signs and symptoms, which may be in connection with proceeding to operation 408.

In view of the foregoing, recommending a procedure involves selecting an appropriate procedure recommendation decision tree even in the absence of a complete diagnosis code, e.g., ICD-10 CM code. Wild cards may be used in the diagnosis code associated with each part of the procedure recommendation decision tree to indicate the broadest set of diagnosis codes that are addressed by that branch of the tree. In order to reach a procedure recommendation, the present disclosure applies guidelines to diagnosis codes and user-entered symptoms in order to differentiate treatment choices. The recommended procedure and associated code may vary according to whether the patient is outpatient or inpatient. For example, the different patient location designations mean that different code sets will be employed. Although the procedure code recommendation may involve complexities because a single outpatient (CPT) code may have more than one inpatient (PCS) code, methods of the present disclosure are adapted to account for these complexities because user involvement in the procedure recommendation flow means that the user may be provided with additional requests for information regarding the patient in order to narrow the selections to a particular procedure code.

Treatment Recommendation Enhancement Routine

According to further implementations, methods for providing treatment recommendation enhancements may be provided. This enhancement routine may involve identifying a complete diagnosis code, for example, according to processes described in connection with FIGS. 2-4. The diagnosis code and the patient's clinical criteria entered by the user may then be used to identify appropriate treatment recommendations for the patient. For example, the diagnostic information and signs and symptoms entered may be used to identify the severity or complexity of a patient's condition, and a recommended level of treatment may be provided based thereon. In a particular example, the number of physical therapy sessions recommended based upon evidence-based medicine or payer policies for rehabilitating a patient experiencing a particular condition may be generated by analyzing the patient's signs and symptoms and related diagnosis code.

Elements of the system of the present disclosure are described with respect to one or more possible embodiments, and are not intended to be limiting. For example, aspects of the described apparatuses may be removed or may be replaced with substitutes that perform similar functions.

Further, aspects of the present disclosure may be provided as a computer program product, or software, that may include, for example, a computer-readable storage medium or a non-transitory machine-readable medium having stored thereon instructions, which may be used to program a computer system (or other electronic devices) to perform a process according to the present disclosure. A non-transitory machine-readable medium may be any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (“ROM”); random access memory (“RAM”); erasable programmable memory (e.g., EPROM and EEPROM); flash memory; and so on.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular embodiments. Functionality may be separated or combined in procedures differently in various embodiments of the disclosure or described with different terminology. These and other variations, modifications, additions, and improvements may fall within the scope of the disclosure and the inventive subject matter.

What is claimed is:

1. A computer-implemented method for recommending a procedure, the method comprising:
using a computer processor to perform the steps of:
receiving at least a partial diagnosis code;
using the at least partial diagnosis code to identify a plurality of clinical criteria associated with the at least partial diagnosis code, the plurality of clinical criteria comprising a plurality of signs and symptoms and stored in a computer memory;
receiving one or more selections of the plurality of clinical criteria;
generating a diagnosis code or a substantially complete diagnosis code using the at least partial diagnosis code and the received one or more selections of the plurality of clinical criteria, wherein the diagnosis code or substantially complete diagnosis code comprises a greater number of digits relative to the at least partial diagnosis code;
identifying one diagnostic procedure recommendation decision tree from a plurality of diagnostic procedure recommendation decision trees stored in the computer memory using the generated diagnosis code or substantially complete diagnosis code, wherein branches of the decision tree are each assigned a different partial diagnosis code, and decision end points of the decision tree are each associated with a different procedure recommendation and corresponding procedure code;
determining a set of clinical criteria required to recommend a procedure using the decision tree and using the selections of the plurality of clinical criteria;
providing additional clinical criteria for selection using the determined set of clinical criteria;
receiving selections from the provided additional clinical criteria selections, the selections associated with a patient's signs and symptoms; and
providing a recommended procedure to administer to the patient and a corresponding procedure code by applying the received selections of additional clinical criteria to the identified decision tree.

2. The method of claim 1, wherein if the step of generating a diagnosis code or a substantially complete diagnosis code results in a substantially complete diagnosis code, the step of providing the recommended procedure comprises identifying a recommended procedures to confirm a complete diagnosis and a corresponding complete diagnosis code.

3. The method of claim 1, wherein if the step of generating a diagnosis code or a substantially complete diagnosis code results in a substantially complete diagnosis code, the processor further performs the step of assigning a default value to the substantially complete diagnosis code.

4. A computer-implemented method for recommending a procedure, the method comprising:
using a computer processor to perform the steps of:
receiving a query for a search for a diagnosis code;
identifying a plurality of at least partial diagnosis codes, stored in a computer memory, using the received query;
receiving a selection of one of the at least partial diagnosis code from the plurality of at least partial diagnosis codes;
identifying a plurality of clinical criteria related to the selected at least partial diagnosis code, the plurality of clinical criteria comprising a plurality of signs and symptoms;
receiving one or more selections of the identified plurality of clinical criteria;
generating a diagnosis code or a substantially complete diagnosis code using the selected at least partial diagnosis code and the received one or more selections of the plurality of clinical criteria, wherein the diagnosis code or substantially complete diagnosis code comprises a greater number of digits relative to the selected at least partial diagnosis code; and
providing a recommended procedure and corresponding procedure code using the generated diagnosis code or substantially complete diagnosis code and the received selections of the plurality of clinical criteria.

5. The method of claim 4, wherein if the step of generating a diagnosis code or a substantially complete diagnosis code results in a substantially complete diagnosis code, the step of providing the recommended procedure comprises identifying a recommended procedure to confirm a complete diagnosis and a corresponding complete diagnosis code.

6. The method of claim 4, wherein if the step of generating a diagnosis code or a substantially complete diagnosis code results in a substantially complete diagnosis code, the processor further performs the step of adding a default value to the substantially complete diagnosis code.

7. The method of claim 4, wherein the step of receiving the query for a search for a diagnosis code comprises receiving at least one of a procedure or a procedure code from a user, and wherein the at least one of the procedure or procedure code is used to identify the plurality of clinical criteria related to the selected at least partial diagnosis code.

8. The method of claim 4, where the identified plurality of partial diagnosis codes comprises one or more of parent codes, primary child codes, and wherein the partial diagnosis codes are 3- to 5-digit codes.

9. The method of claim 4, wherein the clinical criteria further comprises one or more of previous exams, contraindications, safety issues or body parts associated with the partial diagnosis code.

10. The method of claim 4, wherein the plurality of signs and symptoms is an initial set of signs and symptoms, and the plurality of clinical criteria further comprises a further set of clinical criteria, and wherein in response to receiving the one or more selections from the initial set of signs and symptoms, prior to generating the diagnosis code or substantially complete diagnosis code, the processor further performing the steps of:
refining the partial diagnosis code using the received selections from the initial set of signs and symptoms;
identifying the further set of clinical criteria comprising additional signs and symptoms, wherein the additional signs and symptoms have an increased specificity relative to the initial set of signs and symptoms; and
receiving one or more selections of the further clinical criteria.

11. The method of claim 4, wherein the processor further performs the step of:
using the one or more selections of the clinical criteria to generate documentation of a patient's condition.

12. The method of claim 4, further comprising using the generated diagnosis code or substantially complete diagnosis code and the received selections of the plurality of clinical criteria to generate treatment recommendations.

13. A computer-implemented method for recommending a procedure, the method comprising:
using a computer processor to perform the steps of:
receiving at least a portion of a diagnosis code;
identifying one diagnostic procedure recommendation decision tree from a plurality of diagnostic procedure recommendation decision trees stored in a computer memory using the received at least a portion of a diagnosis code, wherein branches of the decision tree are each assigned a different partial diagnosis code, and decision end points of the decision tree are each associated with a different procedure recommendation and corresponding procedure code;
determining a set of clinical criteria required to recommend a procedure using the identified decision tree;
providing clinical criteria selections using the determined set of clinical criteria;
receiving selections of clinical criteria associated with a patient from the provided selections; and
providing a recommended procedure to administer to the patient and a corresponding procedure code by applying the received selections of clinical criteria to the identified decision tree.

14. The method of claim 13, wherein the at least a portion of a diagnosis code is received from a medical record of the patient.

15. The method of claim 13, wherein the method further comprises the steps of:
receiving a query for a search for a diagnosis code; and
identifying a plurality of partial diagnosis codes using the received search, wherein the step of receiving the at least a portion of a diagnosis code comprises receiving a selection from the identified plurality of partial diagnosis codes.

16. The method of claim 13, wherein the step of determining a set of clinical criteria required to recommend a diagnostic task using the identified decision tree comprises retrieving clinical criteria from a medical record of the patient and using the retrieved clinical criteria to determine additional clinical criteria required to recommend the diagnostic task.

17. The method of claim 13, wherein if the applied selections are insufficient to reach a decision end point in the identified decision tree, the step of providing a recommended procedure and corresponding procedure code comprises providing a default procedure and procedure code.

18. The method of claim 13, further comprising the step of determining whether the recommended procedure and corresponding procedure code are accepted, and if the recommendation is not accepted, determining whether another procedure and corresponding procedure code are specified.

19. The method of claim 18, further comprising the step of determining whether the specified another procedure is allowed under an insurance policy of the patient.

20. The method of claim 13, further comprising at least using the received at least partial diagnosis code and the received selections of clinical criteria associated with the patient to generate treatment recommendations for the patient.

21. The method of claim 13, further comprising the step of receiving at least one of a procedure or a procedure code, and wherein the step of identifying the one diagnostic procedure recommendation decision tree from the plurality of diagnostic procedure recommendation decision trees comprises using the received at least one of the procedure or procedure code to identify the one diagnostic procedure recommendation decision tree.
